(19) United States
(12) Patent Application Publication
(10) Pub. No.: US 2010/0048740 A1
(43) Pub. Date: Feb. 25, 2010

(54) Naturally Derived Emulsifier System

(76) Inventors: Michel Mercier, Mountainside, NJ (US); My Trinh Luu, Edison, NJ (US)

Correspondence Address: LOUIS C. PAUL, 420 East 61st Street, 8E, NEW YORK, NY, 10021, US

(21) Appl. No.: 12/595,569

(22) PCT Filed: Apr. 11, 2008
(86) PCT No.: PCT/US08/04705

§ 371 Date: Oct. 12, 2009

Related U.S. Application Data
(60) Provisional application No. 60/907,695, filed on Apr. 13, 2007.

(51) Int. Cl. A61K 8/63; A61Q 19/00

(52) U.S. Cl. 514/785

(57) ABSTRACT

A naturally-derived emulsifier system for use in topically-applied dermatocosmetic products comprising (i) vegetal cholesterol derived semi-synthetically from a plant of the Dioscorea species (D. composite, D. terpinapensis and/or D. villosa) and/or Trillium erectum; (ii) a botanically-derived phospholipid; and (iii) a botanical oil having a fatty acid content of at least 50% by weight of C₁₈ fatty acid(s) having at least two carbon-carbon double bonds.

### FIELD OF INVENTION

The present invention is directed to a naturally-derived emulsifier system for use in topically-applied dermatocosmetic products comprising (i) vegetal cholesterol derived semi-synthetically from a plant of the Dioscorea species (D. composita, D. terpinapensis and/or D. villosa) and/or Trillium erectum, (ii) a botanically-derived phospholipid; and (iii) a botanical oil having a fatty acid content of at least 50% by weight of C₁₈ fatty acid(s) having at least two carbon-carbon double bonds.

### BACKGROUND OF INVENTION

The lamellar structure of the stratum corneum, as well as its role as a permeability barrier, is well-characterized in the scientific and medical literature. See, e.g., Elias P M & D S Friend, “The permeability barrier in mammalian epidermis.” J. Cell. Biol. Vol. 65, pp. 180-191 (1975); Elias P M. “Epidermal lipids, barrier function, and desquamation.” J. Invest. Dermatol. Vol. 80, pp. 44-49 (1983). More particularly, stratum corneum cells are surrounded by intercellular lipids which are mainly composed of cholesterol, ceramides, and free fatty acids. Feingold K R. “The regulation of epidermal lipid synthesis by permeability barrier requirements.” Crit. Rev. Ther. Drug Carrier Syst. Vol. 8, 193-210 (1991). Jackson S M et al. “Pathobiology of the stratum corneum.” West. J. Med. (1993) Vol. 158, pp. 279-285.

Cholesterol is a sterol, a combination of a steroid and an alcohol. Much attention in the scientific and popular literature has been given to the role of cholesterol in causing cardiac and vascular disease. Less well-known, but equally as important, is the role of cholesterol as an integral component in cell membrane function and health, particularly in regulating membrane fluidity. In the cell membrane, cholesterol exists primarily as an ester formed with various fatty acid compounds, rather than as a free alcohol.

The use of cholesterol and modified cholesterol, particularly ethoxylated cholesterol, as emulsifying agents is described in the prior art.

US Patent Application Publication No. 2002/0081322 (assigned to Estée Lauder) discloses a substantially oil-free non-emulsion topically-applied gel composition comprising an aliphatic polyhydric alcohol, an alkali salt of a fatty acid ester, and a carbohydrate-based or sterol-based surfactant/emulsifier. The sterol-based surfactant/emulsifier is further described to be cholesterol or a phytosterol.

U.S. Pat. No. 4,917,891 (assigned to Revlon) discloses a noncomedogenic, oil-in-water emulsion composition free of animal, vegetable and mineral oils, comprising a two-component emulsifier system and volatile silicone fluid. Evaporation of the silicone fluid is taught to leave an essentially oil-free composition on the skin, thereby decreasing the potential for developing acneiform skin conditions. One of the two emulsifier components is taught to be ethoxylated cholesterol. Compositions of the present invention do not comprise ethoxylated cholesterol.

U.S. Pat. No. 4,537,782 (assigned to L'Oreal) discloses gels or emulsoids (defined as emulsions having a liquid dispersing phase and submicron particles) comprising (i) a C₈-C₂₂ fatty acid or a protein condensate of C₈-C₄₀ fatty acid and a polypeptide of animal origin, (ii) a polyoxyethylenated sterol containing 12-40 moles of ethylene oxide, and (iii) a phosphatide. One polyoxyethylenated sterol is identified as cholesterol containing 24 moles of ethylene oxide. Phosphatides are defined to include lecithin of vegetable or animal origin, such as egg lecithin, soy lecithin, colza lecithin, turnsole lecithin or diamidoester of phosphatidylcholine. Compositions of the present invention do not comprise oxyethylenated cholesterol.

U.S. Pat. No. 4,400,295 teaches an emulsifier system formed by reacting the condensation product of a specific fatty acid (i.e., stearic acid, oleic acid or myristic acid) and a basic amino-acid (i.e., arginine, lysine, β-lysine, and hydroxylysine) with a sterol compound, defined to include cholesterol and phytosterols. Compositions of the present invention are not cholesterol reaction products.

The use of cholesterol as an emulsion stabilizer, skin-conditioning agent and viscosity-increasing agent is described in the International Cosmetic Ingredient (“INCI”) Dictionary and Handbook published by the Cosmetic, Toiletry and Fragrance Association.

MMP Inc. offers for sale botanically-derived “vegetal cholesterol” that is produced semi-synthetically from species of Dioscorea (D. composita, D. terpinapensis and D. villosa) and/or Trillium erectum.

“Biomimetic plant sterols” are offered for sale by Laboratoires Sérobiologiques (“LS”) under the following tradenames: Generol 122 N PRL (INCI: Glycine Soja (soy bean) sterol); Generol R (INCI: Brassica campestris (rape seed) sterol); and Phytosoothe (a complex of canola sterols and cetearyl alcohol). According to a February 2007 LS marketing brochure, these biomimetic plant sterol compounds are similar in structure to animal cholesterol, but are obtained from plant sources. The “vegetal cholesterol” of the present invention—one which is produced semi-synthetically from species of Dioscorea (D. composita, D. terpinapensis and D. villosa) and/or Trillium erectum—is identical in structure to cholesterol; it is a mono-unsaturated, secondary alcohol of a cyclopentane-phenanthrene ring structure having the empirical formula C₂₇H₄₆O.

Lecithin is described in the INCI Dictionary as a naturally-occurring mixture of the diglycerides of stearic, palmitic, and oleic acids linked to the choline ester of phosphoric acid. Uses of lecithin reported in the INCI Dictionary include as a skin-conditioning agent and as a surfactant/emulsifying agent. Lecithin, which contains both saturated and unsaturated fatty acids, is isolated and purified primarily from egg yolk and soybeans.

Lecithin may be hydrogenated, such that the unsaturated fatty acids in lecithin are converted to saturated fatty acids via hydrogenation. As described below, hydrogenated lecithin is used in dermatocosmetic products.

Cholesterol is used in skincare products including, for example, Stelatopia Moisturizing Cream from Mustela. The combination of cholesterol, ceramides and phytosphingosine in Stelatopia is claimed to help form an effective skin-hydrating barrier.

Cholesterol is formulated in combination with hydrogenated lecithin in skincare products offered from Awake (Vital Express Lotion Serum A; Direct Nutrition, a moisturizer; Eye Concentrate Mask), Sekkisei (Sekkisei Cream) and Paula's Choice (Hydrating Treatment Cream).

Polysorbate 60 is a mixture of stearate esters of sorbitol anhydrides, consisting predominantly of the monostearate ester, condensed with approximately 20 moles of ethylene oxide. According to the INCI Dictionary, Polysorbate 60 is used as a solubilizer and emulsifier of hydrophobic and hydrophilic systems. The ingredient labeling for Paula's Choice Hydrating Treatment Cream lists cholesterol, hydrogenated lecithin and Polysorbate 60.

Root extracts of Dioscorea (Wild Yam) have been formulated in dermatocosmetic compositions including, for example, L'Oreal Dermo-Expertise Age Perfect Eye Cream for Mature Skin and Murad Age-Balancing Night Cream. The Murad night cream also contains glycine soja (soybean) sterols and lecithin. The use of Dioscorea root extracts in topical products to treat a variety of dermatologic conditions, including for providing moisturization to the stratum corneum and helping to reduce the appearance of fine lines and wrinkles on the face, is described in the patent and scientific literature. See, e.g., listings in INCI Dictionary for D. composite, D. japonica, D. mexicana, D. opposita and D. villosa. See also, US Patent Application Publication No. 2003/0235599. (To the extent pertinent, all published US patent applications and granted US patents cited herein are incorporated in their entirety by reference.)

Oils rich in fatty acids are commonly used in dermatocosmetic products. Corn oil (INCI: Zea mays) comprises approximately 10.6% palmitic acid, 27.3% oleic acid, 53.2% linoleic acid and 1.6% linolenic acid. It is used as an ingredient in a wide range of topical skincare products including from Almay (Hypo-Allergenic Eye Makeup Remover Gel Moisturizing), L'Oreal (Dermo-Expertise RevitaLift Face & Neck Anti-Wrinkle and Firming Cream), Vaseline (Intensive Care Healthy Hand Essentials Moisturizing Lotion), and Dove (Face Care Essential Nutrients Day Cream SPF 15).

Emulsions are among the most common types of topical skincare products. There has been and remains a need for dermatocosmetic products that do not contain animal-derived ingredients. This need is met by the present invention.

### SUMMARY OF THE INVENTION

The present invention relates to a naturally-derived emulsifier system for use in topically-applied dermatocosmetic products comprising (i) vegetal cholesterol derived semi-synthetically from a plant of the Dioscorea species (D. composite, D. terpinapensis and/or D. villosa) and/or Trillium erectum, (ii) a botanically-derived phospholipid; and (iii) a botanical oil having a fatty acid content of at least 50% by weight of C₁₈ fatty acid(s) having at least two carbon-carbon double bonds.

### DETAILED DESCRIPTION OF THE INVENTION

The present invention is directed to a naturally-derived emulsifier system comprising (i) vegetal cholesterol derived semi-synthetically from a plant of the Dioscorea species (D. composita, D. terpinapensis and/or D. villosa) and/or Trillium erectum; (ii) a botanically-derived phospholipid; and (iii) a botanical oil having a fatty acid content of at least 50% by weight of C₁₈ fatty acid(s) having at least two carbon-carbon double bonds.

As used in the present application, by the term “vegetal cholesterol derived semi-synthetically from a plant of the Dioscorea species (D. composite, D. terpinapensis and/or D. villosa) and/or Trillium erectum” is meant a mono-unsaturated, secondary alcohol of a cyclopentanophenanthrene ring structure having the empirical formula C₂₇H₄₆O that is synthesized by a process having cholesterol acetate as an intermediate. The cholesterol acetate is hydrolyzed, producing the claimed vegetal cholesterol.

In one preferred embodiment of the present invention, the vegetal cholesterol is extracted and purified from a plant of the Dioscorea species.

In a further preferred embodiment, the vegetal cholesterol is extracted and purified from a mixture of D. composita, D. terpinapensis and/or D. villosa.

In another preferred embodiment, the vegetal cholesterol is extracted and purified from Trillium erectum.

In yet another preferred embodiment, the vegetal cholesterol is extracted and purified from at least two of D. composita, D. terpinapensis, D. villosa and T. erectum.

The vegetal cholesterol derived semi-synthetically from a plant of the Dioscorea species (D. composita, D. terpinapensis and/or D. villosa) and/or Trillium erectum is present at a concentration of at least about 0.01%, preferably at a concentration of at least about 0.05%, more preferably at a concentration of at least about 0.1% based on the total weight of the emulsifier system. Unless otherwise noted, percentages are by weight of the referenced composition.

A second component of the naturally-derived emulsifier system of the present invention is a botanically-derived phospholipid. As used in the present application, by the term phospholipid is meant a glycerol moiety on which two fatty acids are esterified at the carbon-1 and carbon-2 positions, and a phosphate group is esterified at the carbon-3 position. The fatty acid groups which are typically esterified to the glycerol may be saturated or unsaturated C₁₂-C₂₀ fatty acids, and are preferably selected from the group consisting of palmitic, stearic, oleic, linoleic, linolenic and arachidonic acids.

The phosphate group may be substituted or unsubstituted. In its unsubstituted form, the phospholipid is phosphatidic acid. Substituents on the phosphate group are well-known to those of skill in the art and may include nitrogen-containing alcohols (e.g., ethanolamine, serine and choline) and inositol (hexahydroxycyclohexane).

A preferred botanically-derived phospholipid is lecithin derived from soybeans. The soybean-derived lecithin is present at a concentration of at least about 1%, preferably at a concentration of at least about 1.5%, based on the total weight of the emulsifier system.

A third component of the naturally-derived emulsifier system of the present invention is a botanical oil having a fatty acid content of at least 50% by weight of C₁₈ fatty acid(s) having at least two carbon-carbon double bonds.

Omega-3 fatty acid (also known as alpha-linolenic acid and all-cis-9,12,15-octadecatrienoic acid) is a C₁₈ fatty acid having three cis carbon-carbon double bonds at the carbon-9, carbon-12 and carbon-15 positions. Botanical sources of omega-3 fatty acids include flax seed oil, canola oil, and soybean oil.

Omega-6 fatty acid (also known as gamma-linolenic acid and all-cis 6,9,12-octadecatrienoic acid) is a C₁₈ fatty acid having three cis carbon-carbon double bonds at the carbon-6, carbon-9 and carbon-12 positions.

Linoleic acid (also known as cis-cis-9,12-octadecadienoic acid) is also an omega-6 fatty acid. It is a C₁₈ fatty acid having two cis carbon-carbon double bonds at the carbon-9 and carbon-12 positions. Botanical sources of omega-6 fatty acid include black currant oil, evening primrose oil, borage oil, grape seed oil and corn oil.

Non-limiting examples of botanical oils having a fatty acid content of at least about 50% by weight of C₁₈ fatty acids having two carbon-carbon double bonds include: corn oil, flaxseed oil, safflower oil, soybean oil, cottonseed oil, sunflower oil, grapeseed oil, poppyseed oil, walnut oil, and wheat germ oil. Of these, corn oil is particularly preferred.

Preferably, the botanical oil having a fatty acid content of at least about 50% by weight of C₁₈ fatty acids having two carbon-carbon double bonds is present in the naturally-derived emulsifier system of the present invention at a concentration of at least about 50% based on the total weight of the emulsifier system.

In a particularly preferred embodiment, the naturally-derived emulsifier system of the present invention comprises a botanical oil having a fatty acid content of at least about 50% by weight of C₁₈ fatty acids having two carbon-carbon double bonds and a fatty acid content of at least about 75% by weight of C₁₈ fatty acid(s) having one or two carbon-carbon double bonds.

Oleic acid (also known as 9-octadecenoic acid) is known as an omega-9 fatty acid having one carbon-carbon double bond.

A preferred botanical oil having a fatty acid content of at least about 50% by weight of C₁₈ fatty acids having two carbon-carbon double bonds and a fatty acid content of at least about 75% by weight of C₁₈ fatty acids having one or two carbon-carbon double bonds is corn oil, which has at least about 25% oleic acid and at least about 50% linoleic acid.

The emulsifier system of the present invention may also comprise one or more additional surfactants which are derived and modified from plant sources or are produced synthetically. Such surfactants are well-known to those of skill in the art, and may be amphoteric, anionic, cationic, or non-ionic. Amphoteric surfactants include propionates, alkyldimethyl betaines, alkylamido betaines, sulfobetaines, and imidazoline. Anionic surfactants include fatty alcohol sulfates, alpha olefin sulfonates, sulfosuccinates, phosphate esters, carboxylates and sarcosinates. Cationic surfactants include alkyl quaternaries, alkylamido quaternaries, imidazoline quaternaries. Nonionic surfactants include alkanolamides, ethoxylated amides, esters, alkoxylated alcohols, alkoxylated triglycerides, alkylpolyglucosides, amine oxides, sorbitan esters and ethoxylates. Surfactants may also be silicone surfactants including, but not limited to, dimethicone copolyols, alkyl dimethicone copolyols, silicone quaternary compounds, silicone phosphate esters and silicone esters.

Preferably the additional surfactant that is derived and modified from a plant source or produced synthetically, is non-ionic.

One preferred non-ionic surfactant is Polysorbate 60, a mixture of stearate esters of sorbitol anhydrides, consisting predominantly of the monostearate ester, condensed with approximately 20 moles of ethylene oxide.

Another preferred non-ionic surfactant is glyceryl undecylenate, the ester of glycerin and undecylenic acid.

In a particularly preferred embodiment, the naturally-derived emulsifier system of the present invention is comprised of two non-ionic surfactants, preferably Polysorbate 60 and glyceryl undecylenate.

One aspect of the present invention is directed to use of the naturally-derived emulsifier system of the present invention in emulsions comprised of water phase and one or two phases which are immiscible in water—either oil or silicone. More particularly, the naturally-derived emulsifier system of the present invention may be “dual-phase” with water as the external phase (oil-in-water; silicone-in-water) or with water as the internal phase (i.e., water-in-oil; water-in-silicone). Emulsions of the present invention may also be “three-phase” (e.g., water-in-oil-in-water; oil-in-water-in-oil).

Emulsions comprising the naturally-derived emulsifier system of the present invention may be in the form of a cream, lotion, serum or gel. The naturally-derived emulsifier system of the present invention is typically present at a concentration of at least about 3% by weight of the total emulsion.

The naturally-derived emulsifier system of the present invention is prepared according to principles and techniques well-known to those of skill in the art of formulating topically-applied products. Ingredients which are oil soluble (e.g., Corn Oil or other botanical oils) or dispersible (e.g., botanical sources of omega-3 and omega-6 fatty acids) are mixed together. Preferably mixing is done at 3000 to 4500 rpm for about 15 minutes at a temperature of about 60° C. using a medium speed homogenizer known to those of skill in the art (e.g., Silverson L4RT manufactured by A. Silverson Machines, Ltd., Chesham, England). After 15 minutes, at least one non-ionic surfactant, preferably two, is added with medium propeller mixing. In a preferred embodiment, Polysorbate-60 and glyceryl undecylenate are added at a temperature of from about 50-55° C. and mixed for fifteen minutes. The mixture is then cooled to about 25° C., at which point vegetal cholesterol is added. Mixing continues until the vegetal cholesterol is dissolved.

Another aspect of the present invention is directed to improving or maintaining skin barrier by applying an emulsion comprising the naturally-derived emulsifier system of the present invention. Such an emulsion may comprise one or more ingredients known to those of skin the art to help improve and maintain skin barrier function. Non-limiting examples of such ingredients include ceramides, glycosaminoglycans, and glycosphingolipids. Similarly, the naturally-derived emulsifier system of the present invention may itself contain one or more of ceramides, glycosaminoglycans, and glycosphingolipids.

The INCI Dictionary describes a wide variety of non-limiting cosmetic and pharmaceutical ingredients commonly used in skincare products that are suitable for inclusion in emulsion formulations comprising the naturally-derived emulsifier system of the present invention. Among these are skin-conditioning agents (e.g., humectants, moisturizers and skin conditioners), a wide array of “active” ingredients, including antioxidants/free radical scavengers, hydroxy acids, peptides, vitamins and derivatives, sunscreens, as well as rheological modifying agents, as set out in U.S. Pat. No. 6,492,326, Col. 5, line 35- Col. 20, line 52.

### Examples

The following examples are further illustrative of the present invention. The components and specific ingredients are presented as being typical, and various modifications can be derived in view of the foregoing disclosure within the scope of the invention.

### Example 1

| Phase | Ingredient | INCI | % w/w |
|---|---|---|---|
| A | DI Water | Water | 73.96 |
| | Crystalcast ™ | Beta-Sitosterol & Sucrose Stearate (&) | 5.50 |
| | | Sucrose Distearate (&) Cetyl Alcohol | |
| | | (&)Stearyl Alcohol | |
| | Xanthan Gum | Xanthan Gum | 0.20 |
| B | Clearocast ™ 550 (MMP) | Isododecane (&) Isononyl Isononanoate | 10.00 |
| | Clary Sage Fermented Extract | Sclareolide | 0.02 |
| | (MMP) | | |
| | Clearocast ™ 100 (MMP) | Dimethicone | 5.00 |
| | Vegephos ™ MM (MMP) | Zea Mays (Corn) Oil (&) Polysorbate-60 | 5.00 |
| | | (&) Glyceryl Mono Undecylenate (&) Soy | |
| | | Lecithin (&) Cholesterol | |
| C | Germall Plus Liquid (ISP) | Propylene Glycol (&) Diazolidinyl Urea (&) | 0.30 |
| | | Iodopropynyl Butylcarbamate | |
| | Aromatic Fragrances and Flavor | Flavor | 0.02 |
| | International After Shave Balm | | |
| | frag. #116622 | | |

The following procedure is used to formulate an after shave balm having a pH of 5.57 and a viscosity of 21,500 cps (LV4@12 rpm). Add DI Water to main vessel, combine Phase B in a side vessel. Heating both vessels to 80-85° C. When main vessel temperature reaches 80-85° C., add Crystalcast MM to main vessel, start mixing with propeller agitation at least 15 minutes or until dissolve. Slowly add xanthan gum. Continue mixing. Homogenize for 5 minutes. Add Phase B to main vessel. Hold at temperature of 80-85° C. for 30 minutes. Start cooling to 45° C. When temperature reaches 45° C., switch to sweep blade. Add Phase C to main vessel. Homogenize for 5 minutes at 34° C.

### Example 2

| Ingredient | INCI | % w/w |
|---|---|---|
| Clearocast ™ 550 (MMP) | Isododecane (&) | 77.90 |
| | Isononyl Isononanoate | |
| Clearocast ™ 100 (MMP) | Dimethicone | 10.00 |
| Nayad ® FG (MMP/Immudyne) | Yeast Extract | 0.001 |
| Vegephos ™ MM (MMP) | Zea Mays (Corn) Oil (&) | 10.00 |
| | Polysorbate-60 (&) | |
| | Glyceryl | |
| | Mono Undecylenate (&) | |
| | Soy Lecithin (&) | |
| | Cholesterol | |
| 7-DHC, 1% Sol. In Corn Oil (MMP) | Zea Mays (Corn) Oil & | 1.00 |
| | 7-Dehydrocholesterol | |
| Vitamin E Acetate | Tocopheryl Acetate | 1.00 |
| Shaw Mudge 71040M All Natural | Fragrance | 0.10 |
| Ctirus Lime Peppermint Fragrance | | |

A massage oil is formulated by combine the above-listed ingredients, mixing until clear and then filtering.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A naturally-derived emulsifier system comprising
(i) vegetal cholesterol derived semi-synthetically from one or a mixture of
(a) a plant of the Dioscorea species selected from the group consisting of D. composite, D. terpinapensis and D. villosa
(b) Trillium erectum;
(ii) a botanically-derived phospholipid; and
(iii) a botanical oil having a fatty acid content of at least 50% by weight of C₁₈ fatty acid(s) having at least two carbon-carbon double bonds.

2. The naturally-derived emulsifier system of claim 1 wherein the vegetal cholesterol is derived semi-synthetically from one of D. composita, D. terpinapensis or D. villosa.

3. The naturally-derived emulsifier system of claim 1 wherein the vegetal cholesterol is derived semi-synthetically from Trillium erectum.

4. The naturally-derived emulsifier system of claim 1 wherein the vegetal cholesterol is derived semi-synthetically from two of D. composita, D. terpinapensis, D. villosa, and T. erectum.

5. The naturally-derived emulsifier system of claim 1 wherein the vegetal cholesterol comprises from about 0.05% to about 1.5% by weight of the total emulsifier system.

6. The naturally-derived emulsifier system of claim 1 wherein the botanically-derived phospholipid is soybean lecithin.

7. The naturally-derived emulsifier system of claim 6 wherein the soybean lecithin is present at a concentration of from about 0.05% to about 5% by weight of the total emulsifier system.

8. The naturally-derived emulsifier system of claim 1 wherein the botanical oil having a fatty acid content of at least 50% by weight of C₁₈ fatty acid(s) having at least two carbon-carbon double bonds is present at a concentration of from about 50% to about 90% by weight of the total emulsifier system.

9. The naturally-derived emulsifier system of claim 1 wherein the botanical oil having a fatty acid content of at least 50% by weight of C₁₈ fatty acid(s) having at least two carbon-carbon double bonds is corn oil.

10. The naturally-derived emulsifier system of claim 1 further comprising at least one non-ionic emulsifier.

11. The naturally-derived emulsifier system of claim 10 wherein the at least one non-ionic emulsifier is selected from the group consisting of glycyeryl undecylenate and Polysorbate 60.

12. The naturally-derived emulsifier system of claim 1 comprising two non-ionic emulsifiers.

13. An emulsion comprising the naturally-derived emulsifier system of claim 1 wherein the emulsifier system is at a concentration of from about 4% to about 15% by weight of the total emulsion.

14. The emulsion of claim 13 wherein the emulsion is oil-in-water, silicone-in-water, water-in-oil, water-in-silicone, water-in-oil-in-water or oil-in-water-in-oil.
